(19) United States
(12) Patent Application Publication
(10) Pub. No.: US 2014/0276604 A1
(43) Pub. Date: Sep. 18, 2014

(54) Medical Tube Securing Device

(71) Applicants: Fereshteh K. Maynard, Conway, AR (US); Jeffery B. Wilkerson, Little Rock, AR (US); Victoria L. Eskew, Benton, AR (US)

(72) Inventors: Fereshteh K. Maynard, Conway, AR (US); Jeffery B. Wilkerson, Little Rock, AR (US); Victoria L. Eskew, Benton, AR (US)

(21) Appl. No.: 13/826,468

(22) Filed: Mar. 14, 2013

(51) Int. Cl. A61M 25/02; A61M 27/00

(52) U.S. Cl. CPC A61M 25/02; A61M 27/00
USPC 604/508; 604/174; 604/541; 604/179; 604/180

(57) ABSTRACT

A medical tube securing device including: (a) an anchor that can be attached to the skin of a patient, wherein at least one opening extends through the anchor and is shaped and sized to receive a medical tube; (b) a first padding layer having a first side and a second side, wherein the first side of the first padding layer is joined to the anchor and has at least one opening that extends through the first side and the second side of the first padding layer; (c) at least one clasp joined to the second side of the first padding layer, wherein the clasp receives and holds in place a portion of the medical tube; and (d) a second padding layer. A method of securing a medical tube is also disclosed.

### CROSS-REFERENCE TO RELATED APPLICATIONS

Not applicable

### STATEMENT REGARDING FEDERALLY SPONSORED RESEARCH OR DEVELOPMENT

Not applicable

### BACKGROUND OF THE INVENTION

1. Field of the Invention

The present invention relates to a securing device and, more particularly, to a securing device for medical tubes inserted into a patient's body.

2. Brief Description of the Related Art

Many medical procedures require a drain be inserted into the body of the patient after surgery. Drains are used to prevent the accumulation of fluid and air from the body and thereby promote tissue healing. Laboratory analysis of the drainage fluid may be used for diagnostic purposes. A drain typically includes a tube, which allows passage of the fluid, and a collection vessel for the collection of the fluid that has passed through the tube. Some drains also include a mechanical or manual suction to facilitate the removal of the fluids from the body.

A medical drain is commonly secured in an incision by suturing the skin on both sides of the drain. Medical tape may be used to secure the drain to the patient to further reduce the risk of accidentally pulling or dislodging the drain.

The inventors have observed that these prior art securement methods are often unsuccessful and have to be repeated. The inventors have also observed that these methods are painful and uncomfortable to the patient, and pose a risk of infection and skin breakdown because of repeated removal of tape from the skin.

Most medical procedures, including minor out-patient procedures, require the insertion of an intravenous (IV) line into the patient's arm or hand for delivery of fluids and/or medications to the patient. To start an IV, a catheter or cannula is inserted into a vein. Once inserted, the cannula is secured to the skin of the patient using medical tape to reduce the risk of dislodging. Tubing is attached to the free end of the cannula to deliver the fluids or medications to the patients from the fluid or medication source. The inventors have observed that medical tape is often ineffective in securing the cannula is place and frequent re-taping causes skin breakdown, especially in elderly patients.

It would therefore be desirable to develop a medical tube securing device that is capable of providing stability to the tube inserted into the patient's body, while also protecting and providing comfort to the skin of the patient.

### BRIEF SUMMARY OF THE INVENTION

The present invention is directed to a medical tube securing device comprising: (a) an anchor having a first side and a second side, wherein at least one opening extends through said first side and said second side of said anchor and the opening is shaped and sized to receive a medical tube; (b) a first padding layer having a first side and a second side, wherein said first side of said first padding layer is joined to said second side of said anchor and wherein said first padding layer has at least one opening that extends through said first side and said second side of said first padding layer; (c) at least one clasp joined to said second side of said first padding layer, wherein said at least one clasp receives and holds in place a portion of said medical tube; and (d) a second padding layer.

The present invention is also directed to a method of securing a medical tube having a first end positioned inside a patient's body and a second end positioned outside said patient's body, comprising: (a) passing said second end of said medical tube through an opening of an anchor and an opening of a first padding layer; (b) attaching said anchor to a skin of said patient's body; (c) inserting a portion of said second end of said medical tube into a clasp joined to said first padding layer; and (d) closing said clasp thereby securing said portion of said medical tube in place.

These and other features, objects and advantages of the present invention will become better understood from a consideration of the following detailed description of the preferred embodiments and appended claims in conjunction with the drawings as described following:

### BRIEF DESCRIPTION OF THE DRAWINGS

FIG. 1A is an exploded view of the first preferred embodiment of the present invention.

FIG. 1B is a perspective view of the first preferred embodiment of the present invention.

FIG. 2A is a bottom plan view of the first preferred embodiment of the present invention.

FIG. 2B is a top plan view of the first preferred embodiment of the present invention.

FIG. 2C is a sectional view of the first preferred embodiment of the present invention.

FIG. 3A is a bottom plan view of the second preferred embodiment of the present invention.

FIG. 3B is a top plan view of the second preferred embodiment of the present invention.

FIG. 4A is a perspective view of the second preferred embodiment of the present invention.

FIG. 4B is a perspective view of the first preferred embodiment of the present invention.

### DETAILED DESCRIPTION OF THE INVENTION

With reference to FIGS. 1A-4B, the preferred embodiments of the present invention may be described. The medical tube securing device 10 includes an anchor 12, a padding section 14, and a clasp 16.

The device 10 is secured to the skin of a patient via the anchor 12. As shown in FIGS. 1A-1B, the anchor 12 is preferably square, rectangular, or circular in shape and made of a thin, durable, elastic, hypoallergenic, and breathable material (e.g. polyurethane). The anchor 12 preferably has a top side 20 and a bottom side 22. The bottom side 22 includes a non-skin irritating, hypoallergenic adhesive of a type known to those skilled in the art. The anchor 12, therefore, can be easily attached and removed from the patient's skin without harming the skin. The anchor 12 has an opening 24 that extends through the top side 20 and the bottom side 22 of the anchor. The opening 24 is sized and shaped to receiving a medical tube 26. The medical tube 26 may be JP drain, BLAKE® drain, Davol drain, chest tube, IV line, or any other type of medical drain or tube that would be well-known to those skilled in the art.

The top side 20 of the anchor 12 is attached to the padding section 14. The padding section 14 is preferably made of biodegradable foam. The padding section 14 preferably includes two layers of padding. As shown in FIGS. 2A-2B and FIGS. 3A-3B, the two layers of padding may either be formed by two separate pieces of padding or one piece of padding folded in half to form two layers of padding. The two layers of padding may be described as a first padding layer 28 and a second padding layer 30. The first padding layer 28 remains attached to the anchor 12 while the second padding layer is not directly attached to the anchor 12. The first padding layer 28 has an opening 36 that extends through the padding and abuts the opening 24 of the anchor 12. In other words, the opening 24 in the anchor and the opening 36 in the first padding layer 28 are aligned, thus allowing the tube 26 to pass through both openings 24, 36. In one embodiment, the opening 36 receives a port 32 that is cylindrical in shape and made of plastic. The cylindrical opening of the port 32 is sized and shaped to receive the tube 26.

As shown in FIGS. 1A-1B, the clasp 16 is attached to the inside surface of first padding layer 28. The clasp 16 preferably includes two interlocking pieces and is of a type well-known to those skilled in the art. The clasp 16 receives the tube 26 and locks it in place. Once the tube 26 has been secured in the clasp 16, the second padding layer 30 is either folded over on top of the first padding layer 28 (if the padding section 14 is one piece of padding) or placed on top of the first padding layer 28 (if the padding section 14 is two separate pieces). Thus, the tube 26 is positioned between the two padding layers 28, 30 as shown in FIG. 1B.

As shown in FIG. 2C, in one embodiment, the outside surface of the second padding layer 30 is attached to a backing 34. The backing 34 is preferably made of cardboard. The surface of the backing 34 is suitable for recording medical information, such as the date the drain or tube 26 was inserted.

The device 10 also includes one or more sets of connecting straps 18. The first end of the connecting strap is preferably attached to the second padding layer 30 or to the backing 36. The second end of the connecting strap 18 is attached to the anchor 12. The connecting straps 18 serve to secure the first padding layer 28 and second padding layer 30 together. The two ends of the connecting strap 18 are preferably complementary pieces of VELCRO®. In the embodiment where the padding section 14 is made of two separate pieces instead of one padding piece folded over to form two layers, the device includes two sets of connecting straps positioned on opposite sides of the device as shown in FIGS. 3A-4A.

After certain medical procedures, such as open heart surgery, it is necessary for patients to have multiple drains which are often located in close proximity to one another. Instead of using multiple single-tube devices as described above, in a second preferred embodiment, the device 10 is capable of securing two tubes 26. In this embodiment, as shown in FIGS. 3A-4A, the anchor 12 has two openings 24 and the first padding layer 28 has two openings 36. The two openings 36 of the first padding layer 28 receive the two tubes 26. In one embodiment, the two openings 36 receive two ports 32 which receive the two tubes 26. Two clasps 16 secure the tubes 26 between two layers of padding 28, 30 as described above.

In one application of the present invention as shown in FIG. 4B, the device 10 is used to secure an IV line in a patient 38. Once the catheter or cannula is inserted into the arm or hand of the patient, the free end of the catheter or cannula is passed through the opening 24 of the anchor 12 and the first padding layer 28 (and the port 32 if included). The bottom side 22 of the anchor 12 is then placed and pressed onto the skin of the patient which secures the anchor 12 into place. The IV tubing may then be connected to the free end of the cannula. The cannula and the connected tubing 26 are then inserted in the clasp 16 and the clasp 16 is closed to secure the cannula and tubing into place. The second padding layer 30 is then folded over on the first padding layer 28. The first end of the connecting strap 18 is connected to the second end of the connecting strap 18, thereby securing the two padding layers 28, 30 together.

In a second application of the present invention as shown in FIG. 4A, the device 10 is used to secure and comfortably support two chest tubes 26 in a patient 38. Once the chest tubes 26 are inserted in the patient's chest, the chest tubes are passed through the openings 24 of the anchor 12 and the first padding layer 28 (and the ports 32 if included). The bottom side 22 of the anchor 12 is then placed and pressed on the skin of the patient 38 which secures the anchor into place. The portion of the chest tubes extending directly from the patient's chest are then inserted in the clasps 16 and the clasps 16 are closed to secure the chest tube into place. The second padding layer 30 is then placed on the first padding layer 28. The first ends of the connecting straps 18 are connected to the second end of the connecting strap 18, thereby securing the two padding layers 28, 30 together. In addition to securing the chest tubes, the device 10 provides comfort to the patient by preventing the rigid chest tubes from resting directly on tender tissue after surgery.

The medical tube securing device 10 of the present invention may be used with any type of medical tube or drain in any part of the body, including but not limited to the hand, chest, and leg. Because medical drains and tubes vary in size and in placement, the size and shape of components of the device 10 will also vary accordingly.

The present invention has been described with reference to certain preferred and alternative embodiments that are intended to be exemplary only and not limiting to the full scope of the present invention as set forth in the appended claims.

We claim:

1. A medical tube securing device, comprising:
(a) an anchor having a first side and a second side, wherein at least one opening extends through said first side and said second side of said anchor and said at least one opening is shaped and sized to receive a medical tube;
(b) a first padding layer having a first side and a second side, wherein said first side of said first padding layer is joined to said second side of said anchor and wherein said first padding layer has at least one opening that extends through said first side and said second side of said first padding layer;
(c) at least one clasp joined to said second side of said first padding layer, wherein said at least one clasp receives and holds in place a portion of said medical tube; and
(d) a second padding layer.

2. The medical tube securing device of claim 1, further comprising a connecting strap for securing said first padding layer to said second padding layer.

3. The medical tube securing device of claim 1, wherein said first side of said anchor comprises an adhesive and is capable of being attached to a skin of a patient.

4. The medical tube securing device of claim 1, wherein said medical tube is one of a JP drain, a Davol drain, a chest tube, and an intravenous line.

5. The medical tube securing device of claim 1, wherein said at least one opening of said first padding layer receives at least one port.

6. The medical tube securing device of claim 5, wherein said port receives said medical tube.

7. The medical tube securing device of claim 1, wherein said second padding layer is joined to a backing.

8. The medical tube securing device of claim 2, wherein said connecting strap is comprised of a first piece and a second piece.

9. The medical tube securing device of claim 8, wherein said first piece of said connecting strap is joined to said anchor and said second piece of said connecting strap is joined to said second padding layer.

10. The medical tube securing device of claim 1, wherein said first padding layer and said second padding layer are joined via a folded edge.

11. The medical tube securing device of claim 1, wherein said at least one opening in said anchor comprises two openings and wherein said at least one opening in said first padding layer comprises two openings.

12. A method of securing a medical tube having a first end positioned inside a patient's body and a second end positioned outside said patient's body, comprising:
(a) passing said second end of said medical tube through an opening of an anchor and an opening of a first padding layer;
(b) attaching said anchor to a skin of said patient's body;
(c) inserting a portion of said second end of said medical tube into a clasp joined to said first padding layer; and
(d) closing said clasp thereby securing said portion of said medical tube in place.

13. The method of claim 12, wherein said medical tube is one of a JP drain, a Davol drain, a chest tube, and an intravenous line.

14. The method of claim 12, further comprising the step of attaching a second padding layer to said first padding layer via a connecting strap.
